(19) United States
(12) Patent Application Publication
(10) Pub. No.: US 2018/0333541 A1
(43) Pub. Date: Nov. 22, 2018

(54) Automatic Guard For Syringes

(71) Applicant: Bi-Link Metal Specialties, Inc., Bloomingdale, IL (US)

(72) Inventor: Frank J. Ziberna, Bloomingdale, IL (US)

(73) Assignee: Bi-Link Metal Specialties, Inc., Bloomingdale, IL (US)

(21) Appl. No.: 15/600,198

(22) Filed: May 19, 2017

(51) Int. Cl. A61M 5/32

(52) U.S. Cl. CPC A61M 5/326; A61M 5/3271; A61M 5/3137; A61M 2005/3261; A61M 2005/3263

(57) ABSTRACT

A needle guard for a syringe, includes a base portion having a syringe connection for connecting a syringe and a cover portion and a base portion. The base portion and cover portion are engaged to expose the needle in a use position for administering the syringe to a patient, and after use, the cover portion is configured to slide on the base portion to an extended position on the base portion to cover the otherwise exposed point of the needle in a deployed position. A spring is arranged between the base portion and the cover portion to urge the cover portion to the deployed position on the base portion. An engagement arrangement between the base portion and the cover portion latches the base portion and the cover portion in the use position, and after use, in the deployed position.

### BACKGROUND

Hypodermic syringes are usually administered by a medical caregiver to a patient. A typical syringe includes a cylindrical barrel and a plunger for administering a drug to a patient or drawing a blood sample from a patient. The typical syringe has a fluid-conveying needle covered with a protective, temporary cap. In use, the cap is removed and the needle inserted into a patient. After use, the cap can be replaced on the needle to avoid inadvertent sticking of medical personnel and both are discarded.

Sometimes there can be an accidental sticking of medical personnel during the replacing of the cap. If the cap is not accurately placed onto the needle the nurse or other medical caregiver can be accidentally stuck with the needle. Needless to say, this can be detrimental to the nurse or other medical caregiver given the existence of serious blood borne diseases.

Examples of caps for hypodermic syringe needles are shown in U.S. Pat. Nos. 2,408,323; 2,571,653; 3,073,306; 3,527,216; 3,890,971; 4,355,822; 4,373,526; 4,425,120; and 4826488.

The present inventor has recognized the need for a needle guard that reduces the risk of accidental needle puncture of medical personnel. The present inventor has recognized the need for a needle guard that is easy to use and which is automatically deployed to cover contaminated needles immediately after use.

### SUMMARY

An exemplary embodiment of the invention provides a needle guard for a hypodermic syringe that automatically deploys when the needle is withdrawn from a patient. The invention provides a spring-loaded needle guard that exposes the needle before inserting into a patient and immediately upon withdrawing, the needle guard deploys to cover the exposed needle.

The exemplary embodiment of the invention provides a needle guard that includes a base portion and a cover portion. The base portion and cover portion are engaged to expose the needle in a use position, and after use, the cover portion slides on the base portion to cover the otherwise exposed end or point of the needle.

In one embodiment, the base portion has a socket for connecting a syringe and an engagement portion extending from the socket and having a through channel for receiving the needle. The cover portion substantially surrounds the base portion and has an end opening for allowing the needle that extends out of the through channel to be exposed out of the guard in the use position. The through channel extends through the socket and the engagement portion. The engagement portion includes a catch near the socket and a notch near a distal end of the engagement portion.

The cover portion includes a hook portion that fits into the catch and engages with the catch to be held thereby. The hook portion is biased to move into disengagement with the catch when the cover portion is slid backward with respect to the base portion toward the socket. A spring biases the cover portion away from the socket. When the hook portion is released from the catch, the spring moves the cover portion away from the socket to expand the length of the guard and cover the otherwise exposed needle point. The cover portion slides forward with respect to the base portion until the hook portion engages the notch in the base portion which latches the cover portion to the base portion in a deployed position covering the needle point.

The exemplary embodiment needle guard reduces the risk of accidental needle puncture of medical personnel. The exemplary embodiment needle guard is easy to use and which is automatically deployed to cover contaminated needles immediately after use. The exemplary embodiment needle guard can be effectively used by a medical caregiver with one hand.

Numerous other advantages and features of the present invention will become readily apparent from the following detailed description of the invention and the embodiments thereof, and from the accompanying drawings.

### BRIEF DESCRIPTION OF THE DRAWINGS

FIG. 1 is a perspective view of a needle guard assembly of the present invention mounted on a syringe needle in a ready-to-use position;

FIG. 2 is a sectional view taken generally long line 2-2 of FIG. 1 in a subsequent, position of use just after the ready-to-use position;

FIG. 2a is a fragmentary enlarged view of a portion of FIG. 2 but in the ready-to-use position;

FIG. 3 is a perspective view of the needle car of FIG. 1 mounted on a syringe in a guard deployed position;

FIG. 4 is a sectional view taken generally long line 4-4 of FIG. 3;

FIG. 5 is a perspective view of a base portion of the needle guard assembly shown in FIG. 1;

FIG. 6 is a sectional view taken generally along line 6-6 of FIG. 5;

FIG. 7 is a perspective view of a cover portion of the needle guard shown in FIG. 1;

FIG. 8 is a fragmentary enlarged perspective view of the cover portion shown in FIG. 7.

### DETAILED DESCRIPTION

While this invention is susceptible of embodiment in many different forms, there are shown in the drawings, and will be described herein in detail, specific embodiments thereof with the understanding that the present disclosure is to be considered as an exemplification of the principles of the invention and is not intended to limit the invention to the specific embodiments illustrated.

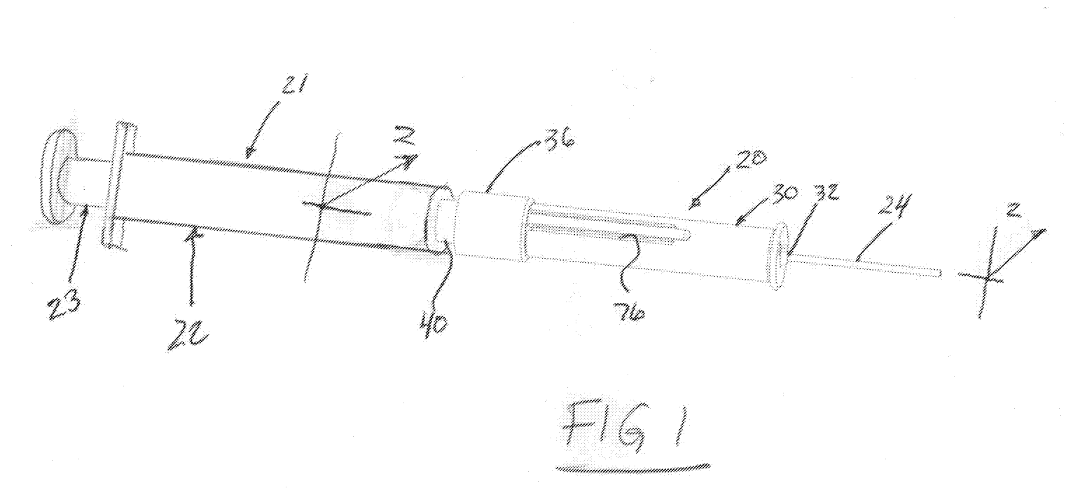

FIG. 1 illustrates a needle guard assembly 20 mounted on a syringe 21. The syringe has a barrel 22 and a plunger 23, a syringe needle 24 with a point 24a, and a syringe hub 40. The point 24a is shown squared off only for purposes of illustration. The assembly 20 includes a base portion 26 engaged to a cover portion 30. The base portion 26 includes a rear socket 36 that receives the syringe hub 40 of the syringe 24. The connection between the socket 36 and the syringe hub 40 can take the form of a Leur Lock connection, a frictional connection, a threaded connection or other known connections. The cover portion 30 includes a front opening 32 allowing the syringe needle 24 to extend through the opening 32 and out of the assembly 20.

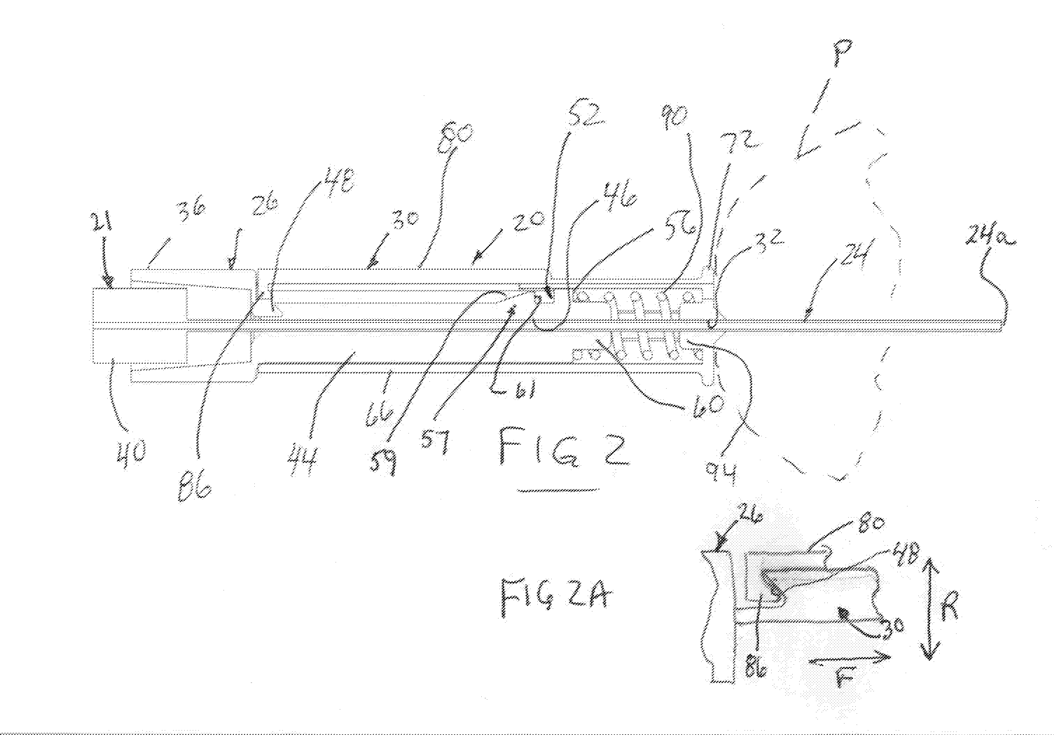

FIG. 2 shows the assembly 20 mounted on a syringe 21 in more detail. Only the hub 40 and the needle 24 of the syringe are shown, the syringe barrel 22 and plunger 23 are not shown. The needle is shown in use, penetrating a patient P. The base portion 26 extends forwardly into an engagement portion 44. The engagement portion 44 has a through channel or path 46 for receiving a portion of length of the syringe needle 24 therein. The engagement portion 44 also includes a hook-shaped catch 48 at a base end and a notch 52 at a distal end. The notch is formed by a rear facing surface 63 of a shoulder 56 at a front side and a ramp 57 having an inclined surface 59 and a vertical or radial plane surface 61 at a rear side. The shoulder 56 is contiguous with a reduced diameter end portion 60 on a front side thereof.

The cover portion 30 comprises a substantially cylindrical body 66 having a disc-shape end plate portion 72 through which opening 32 extends. The body 66 includes an elongated slot 76 over which is arranged a flexible, cantilevered catch arm 80. The catch arm 80 is molded to the body 66 at a base end and includes a hook portion 86 at a free, distal end, which is shaped to interlock within the catch 48. The catch arm 80 is resiliently biased to the natural position shown in FIG. 2, with the hook portion 86 slightly above the catch 48.

A spring 90 is coiled around a portion of the length of the syringe needle 24 and braced between the shoulder 56 of the base portion and the plate portion 72 of the cover portion 30. The plate includes a boss 94 formed as part of the plate portion 72. The spring is guided and restrained at its opposite ends by the boss 94 and the reduced diameter end portion 60, both fit within the spring. The length of this spring is shown after being compressed from its natural length, in FIG. 2.

FIG. 2A shows the interlocking relationship of the hook portion 86 and the catch 48 when the assembly is in the ready-to-use position. The hook portion 86 underlies a portion of the catch 48 and is restrained from moving in the forward direction F and in the radial direction R with respect to the base 26. The hook portion 86 cannot move in the rear direction with respect to the base 26 due to the bias of the spring 90, unless a force is applied.

In FIG. 2, the syringe needle 24 is placed into a patient P to the extent that the plate portion 72 of the cover portion 30 is pressed against the patient. Force on the syringe in the forward direction forces the socket 36 toward the patient and the cover portion 30 moves rearward relative to the base portion 26 a slight distance, given the plate portion abutting the patient, releasing the hook portion 86 from the catch 48 as shown in FIG. 2. Because of the resilient bias of the catch arm 80, the hook portion 86 springs radially away from the base portion (upward in FIG. 2) to its natural position of noninterference with the catch 48. The cover portion 30 is now free to move forwardly with respect to the base portion 26. The spring 90 urges a forward movement of the cover portion 30 with respect to the base portion 26 (in a direction away from the socket 36).

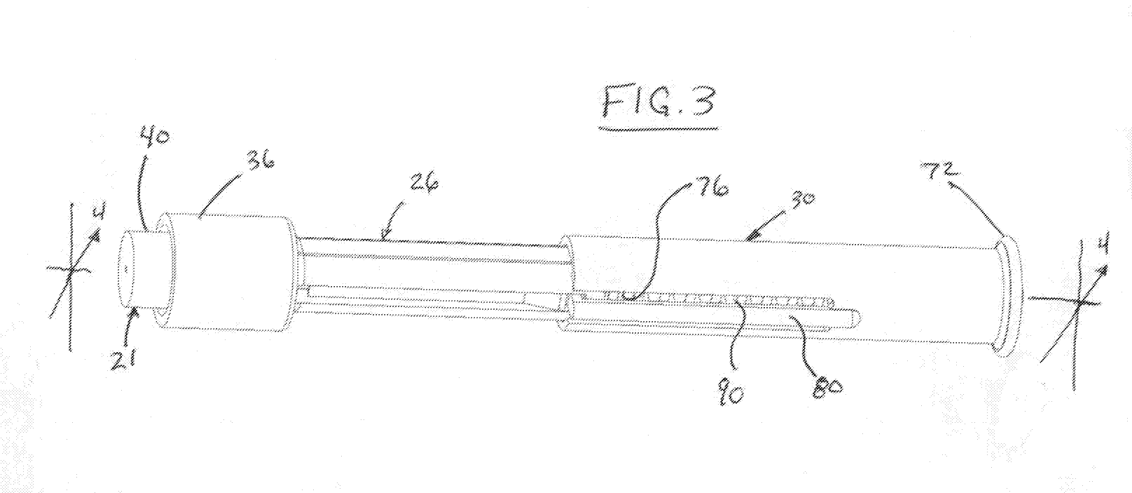

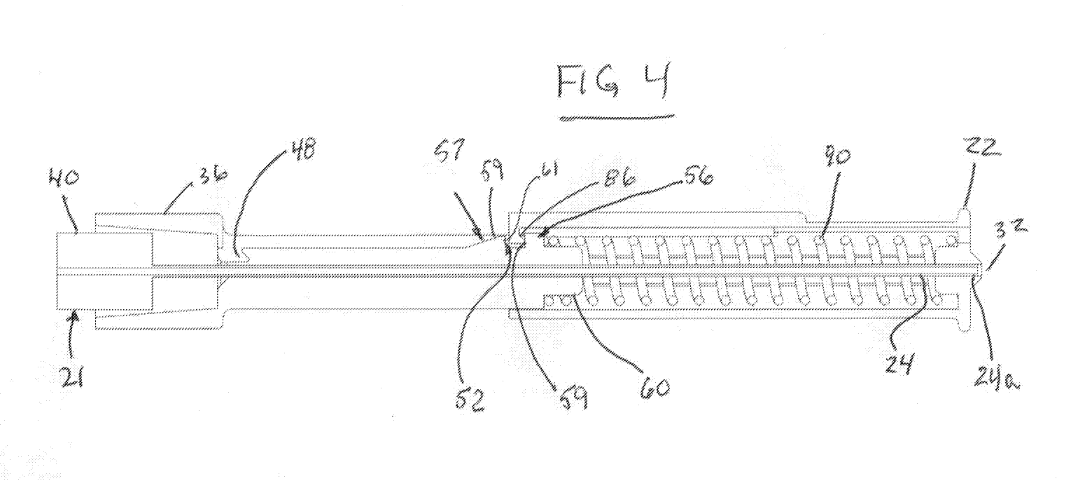

FIGS. 3 and 4 illustrate the relative position of the cover portion 30, the base portion 26 and the syringe needle 24 after the syringe needle 24 has been withdrawn from the patient. The spring 90 has expanded toward its natural length to move the base portion 26 relatively to the left and the cover portion 30 relatively to the right as shown in FIG. 3. By this movement, the length of the guard has been expanded and the entire exposed length of the syringe needle 24 is covered by the cover portion 30 to prevent accidental sticking of a medical worker by the otherwise exposed point 24a of the syringe needle 24.

During movement from the use position (FIG. 2) and then to the deployed position (FIG. 3), the hook portion 86 of the cover portion 30 is displaced resiliently from its normal orientation in a radial direction away from the syringe needle 24 by sliding up the inclined surface 59 of the ramp 57 of the base portion 26 until it snaps radially inwardly into the notch 52 of the base portion 26. The hook portion 86 is thus captured between the radial plane surface 61 and the rear facing surface 63 of the notch 52. The cover portion 30 is thus latched into this orientation with the base portion 26 as shown in FIG. 4.

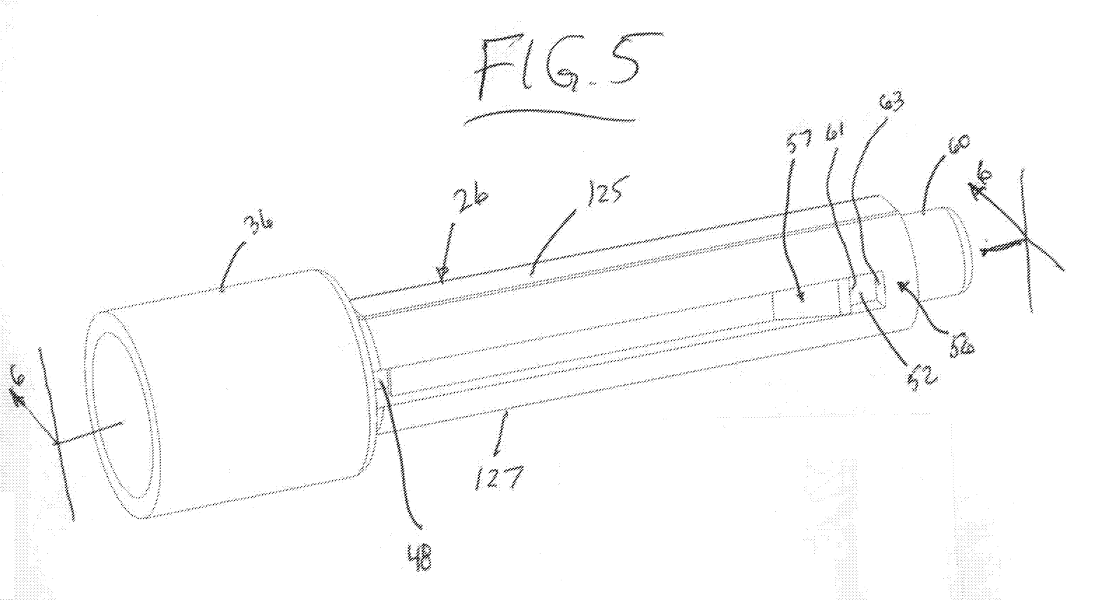

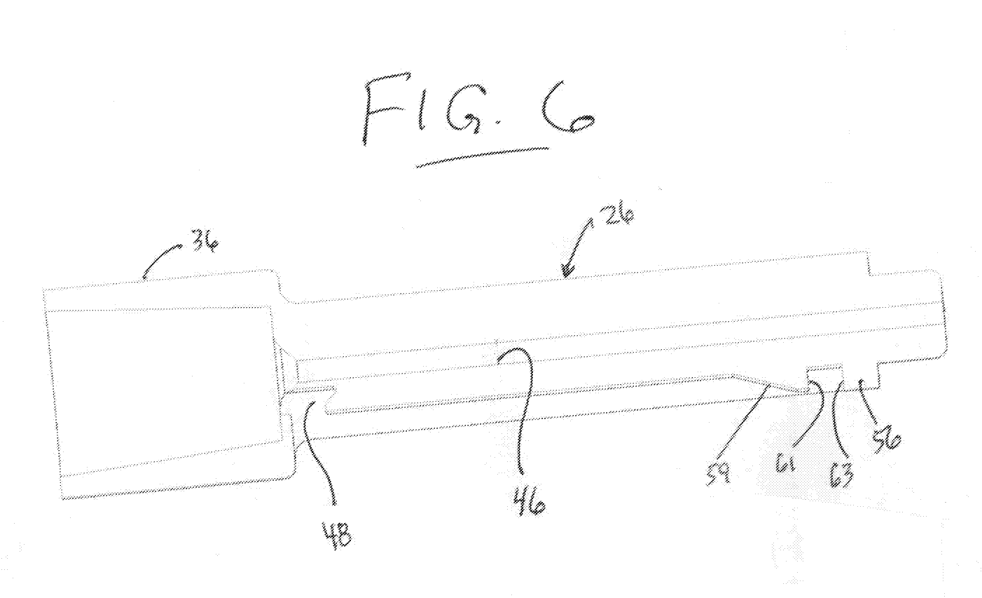

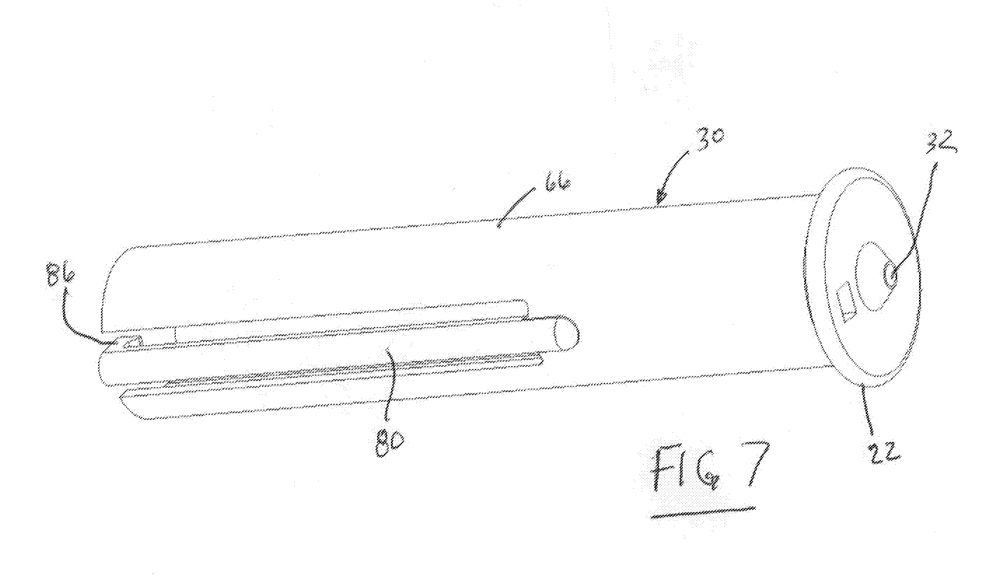

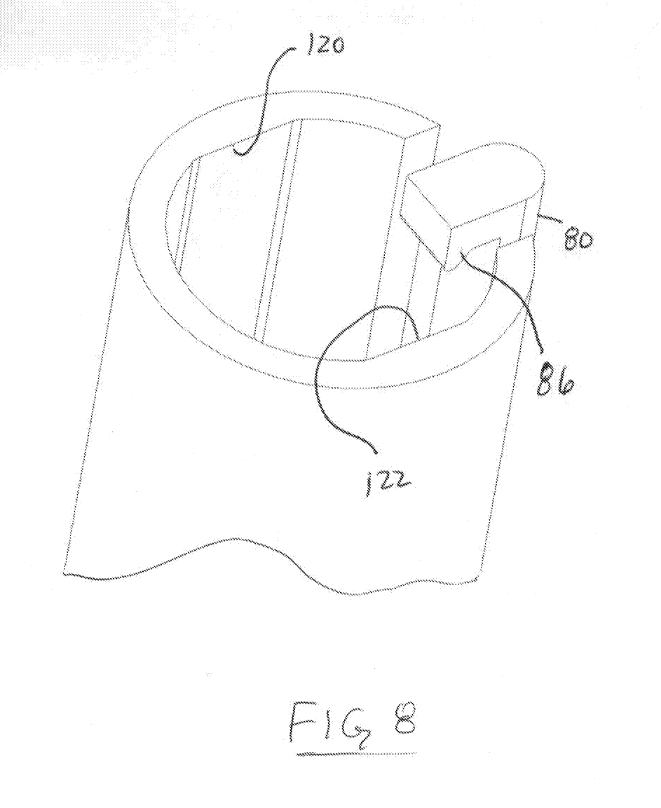

FIGS. 5 through 8 illustrate individual components of the assembly 20 with the syringe 21 removed. FIG. 8 shows that the inside surface of the cover 30 has flat surfaces 120, 122. The base portion 26 has corresponding flat outside surfaces 125, 127. The cooperation of the flat surfaces 120, 122, 125, 127 ensure proper orientation during assembly of the parts 26, 30, and prevents relative rotation between the parts 26, 30 during operation.

In operation, the syringe 21 has the guard 20 installed onto the syringe by connection of the syringe hub 40 and the socket 36, with the syringe needle exposed. The exposed syringe needle has a cap (not shown) that covers the point 24a until ready to use. After the cap is removed, the medical caregiver forces the needle into a patient until the plate portion 72 presses against the surface of the patient. Further pressing displaces the base portion 26 forward a slight distance with respect to the cover portion 30 which springs the hook portion 86 radially to its relaxed position out of engagement with the catch 48. As the syringe is withdrawn from the patient, under force from the spring 90, the base portion 26 slides rearward with respect to the cover portion 30 until the hook portion 86 snaps into the notch 52 to lock the guard covering the needle point 24a.

The materials of construction of the needle guard can be polypropylene or other suitable material for the base portion and the cover portion. The spring can be of known material for a medical device.

The embodiment of the present invention provides a needle guard that is spring loaded to automatically deploy to cover the needle point after the needle is withdrawn from the patient. The guard permits one handed operation which further reduces accidental needle sticks of medical caregivers.

From the foregoing, it will be observed that numerous variations and modifications may be effected without departing from the spirit and scope of the invention. It is to be understood that no limitation with respect to the specific apparatus illustrated herein is intended or should be inferred.

The invention claimed is:

1. A needle guard for a syringe, comprising:
a base portion and a cover portion, the base portion having a syringe connection for connecting a syringe;
the base portion and the cover portion are engaged to expose the needle in a use position for administering the syringe to a patient, and after use, the cover portion is configured to slide on the base portion to a deployed position to cover the otherwise exposed point of the needle;
a spring arranged between the base portion and the cover portion to urge the cover portion to the deployed position on the base portion; and
an engagement arrangement between the base portion and the cover portion to latch the base portion and the cover portion in the use position, and after use, to latch the base portion and the cover portion in the deployed position.

2. The guard according to claim 1, wherein the engagement arrangement comprises a hook portion arranged on one of the base portion and the cover portion, and a catch arranged on the respective other of the base portion and the cover portion, wherein engagement of the hook portion to the catch acts to latch the cover portion in the use position on the base portion.

3. The guard according to claim 2 wherein the hook portion is formed onto the end of a cantilever arm that resiliently extends from the cover portion and the catch is formed on the base portion.

4. The guard according to claim 3, wherein the engagement arrangement comprises a notch arranged spaced from the catch on the base portion, wherein the hook portion engages the notch when the cover is in the deployed position.

5. The guard according to claim 4, wherein the cover portion is substantially cylindrical and surrounds the base portion, the base portion having a passage that receives the needle and the cover portion has an opening to receive the needle and expose the needle at a front of the guard.

6. The guard according to claim 5, wherein the syringe connection comprises a socket for engaging a syringe hub.

7. The guard according to claim 6, wherein the notch is defined by a ramp on a rear side and a radial plane surface on the front side.
